(19) United States
(12) Patent Application Publication
(10) Pub. No.: US 2022/0147673 A1
(43) Pub. Date: May 12, 2022

(54) FATIGUE SCREENING METHOD

(71) Applicant: FMC Technologies, Inc., Houston, TX (US)

(72) Inventors: Nigel Mckie, Houston, TX (US); Carlos Silva, Houston, TX (US)

(73) Assignee: FMC Technologies, Inc., Houston, TX (US)

(21) Appl. No.: 17/091,972

(22) Filed: Nov. 6, 2020

(51) Int. Cl. G06F 30/28; G01N 11/00

(52) U.S. Cl. CPC G06F 30/28; G06F 30/23; G01N 11/00

(57) ABSTRACT

A method includes generating a 3D computer-coded model of a component and performing simulations on the model to determine an onset of gross plastic deformation in a plurality of regions of the component, wherein the model is stored in a computer-readable medium.

### BACKGROUND

Conventional fatigue analysis for components used in the oil and gas industry are generally based on SN-curves. Such SN-curves are plots of stress (load) on a material versus the number of cycles to failure for a given material. SN-curves are often developed by subjecting a sample (i.e., a coupon) of a material to a cyclic stress until a failure occurs in the coupon. Several coupons are tested to develop an SN-curve for the material. However, real components in oil and gas applications are often imperfect and may have more cracks than such coupons that are accurately manufactured and used to generate the SN-curves. For this reason, fatigue failure trendlines based on conventional analysis are often shifted to an extent that the analysis becomes inaccurate.

For example, conventional fatigue analysis generally predicts that threads such as ACME threads of a component would have short a fatigue live. A conventional SN approach, which is usually applicable to high cyclic applications, assumes that a highly concentrated stress over a small region is applied over the entire body of the component. This makes the analysis difficult to account for stress gradients exhibited by threads of the component. Conventional fracture mechanics can account for stress gradients, but does not provide accurate results in the small crack regime. This forces the modeling of a crack propagation to begin with a relatively large crack, and results of such simulations where the crack is not properly sized are often inaccurate.

### SUMMARY

In one aspect, embodiments of the present disclosure relate to methods that include generating a 3D computer-coded model of a component and performing simulations on the model to determine an onset of gross plastic deformation in a plurality of regions of the component, wherein the model is stored in a computer-readable medium.

In another aspect, embodiments of the present disclosure relate to methods that include determining an onset of gross plastic deformation of a component by performing simulations on a 3D computer-coded model of a plurality of regions of the component, and dividing the onset of gross plastic deformation by a safety factor to calculate a working capability load of the model.

Other aspects and advantages of the claimed subject matter will be apparent from the following description and the appended claims.

### BRIEF DESCRIPTION OF DRAWINGS

The following is a description of the figures in the accompanying drawings. In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. For the sake of continuity, and in the interest of conciseness, same or similar reference characters may be used for same or similar objects in multiple figures. Further, the particular shapes of the elements as drawn are not necessarily intended to convey any information regarding the actual shape of the particular elements and have been solely selected for ease of recognition in the drawing.

FIG. 1 shows a check valve model in accordance with one or more embodiments.

FIG. 2 shows a load vs. displacement graph of a simulation in accordance with one or more embodiments.

FIG. 3 shows a simulation result of a check valve in accordance with one or more embodiments.

FIG. 4 shows a simulation result of a check valve in accordance with one or more embodiments.

FIG. 5 shows a simulation result of a check valve in accordance with one or more embodiments.

FIG. 6 shows a simulation result of a check valve in accordance with one or more embodiments.

FIG. 7 shows a flow chart of performing simulations on a component in accordance with one or more embodiments.

FIG. 8A shows a computing system in accordance with one or more embodiments.

FIG. 8B shows a network in accordance with one or more embodiments.

FIG. 9 shows a cross-sectional view of a threaded cap connection of a check valve in accordance with one or more embodiments.

FIG. 10 shows a load vs. deformation graph of a material in accordance with one or more embodiments.

### DETAILED DESCRIPTION

In the following detailed description, certain specific details are set forth in order to provide a thorough understanding of various disclosed implementations and embodiments. However, one skilled in the relevant art will recognize that implementations and embodiments may be practiced without one or more of these specific details, or with other methods, components, materials, and so forth. In other instances, well known features or processes have not been shown or described in detail to avoid unnecessarily obscuring descriptions of the implementations and embodiments.

Embodiments of the present disclosure relate generally to assessment and performance prediction of components used in oil and gas applications by simulating performance of the components (e.g., using finite element analysis (FEA)) in operating conditions and comparing results with real performance of the components. Unlike conventional fatigue failure analyses, methods disclosed herein may include determining an Onset of Gross Plastic Deformation (OGPD) for analyzing performance of a component.

As used herein, OGPD refers to the point at which a component or a portion of a component transitions from elastic behavior to plastic behavior as a load is applied. The OGPD may be determined using a load vs. displacement graph (i.e., a graph showing the amount of stress applied to a component or a selected portion of the component versus the amount of displacement or change in size). The component is said to have elastic behavior when the relationship between load and displacement is linear, and plastic behavior when the relationship is non-linear. OGPD is analogous to yield point. However, unlike the yield point, which is specific to a material, the OGPD is specific to a particular assembly, component, or a selected portion of a component (e.g., threads of a screw-type connection in a component).

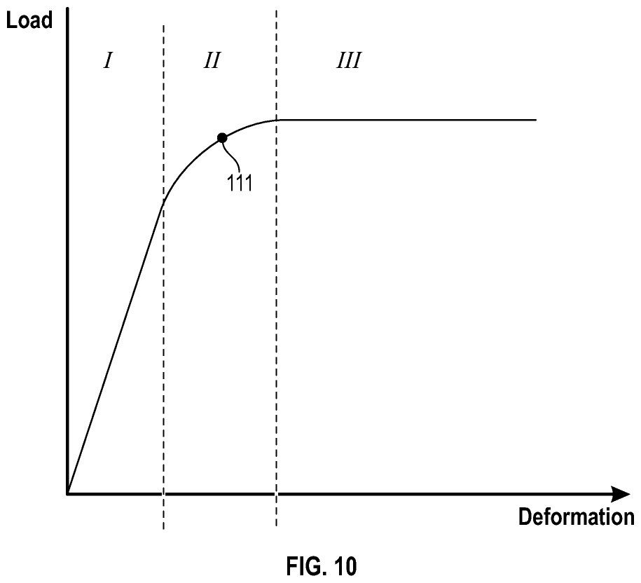

To explain some of the aforementioned terms in more details, FIG. 10 illustrates a general load vs. deformation graph of a component where the graph is divided into sections I, II, and M. Section I shows a perfectly-linear relationship between an increase in load and deformation of the component. The component is said to be in elasticity-dominated deformation region until the material reaches a point 111. This particular point 111 is the OGPD and located in section II which begins from a local yielding of the component. The OGPD 111 approximately demarcates the elasticity-dominated region and the plasticity-dominated region over the entire load vs. deformation graph. Section III shows a perfectly-plastic behavior of a material where there is no increase in load in response to deformation of the component.

The OGPD, in accordance with one or more embodiments, may be defined when a parameter across the weakest region of a component exceeds a maximum contour value obtained by an equation, (yield strength/Young's Modulus)×0.999. The weakest region of a component may be selected as, for example, a region of the component that historically has showed relatively higher failure rate during operation, a region of the component subjected to the highest amount of stress, or a region of the component predicted to fail first based on parameters such as size, shape and material of the region. For example, in embodiments including analysis of a threaded connection in a component, the threads may be selected as the weakest region of the component, and the OGPD may be defined when a parameter across the threads exceeds a maximum contour value. In accordance with one or more embodiments, the parameter may be a change in slope of a stress-strain curve during simulations, or may be a value from analyzing the simulation results in some other embodiments.

Further, in accordance with one or more embodiments, an OGPD may be divided by a safety factor to calculate a working capability of a component. As discussed in more detail below, a component model may be simulated by defining a plurality of parameters of the component, such as boundaries (size/shape) and material characteristics, and demarking a plurality of regions throughout the component model. The model may be used to check the safety of the component by ensuring that all of the OGPD of the plurality of regions of the component are above excessive deformation criterion.

The OGPD may be used to evaluate different failure modes of a component, including excessive deformation, plastic collapse, and fatigue.

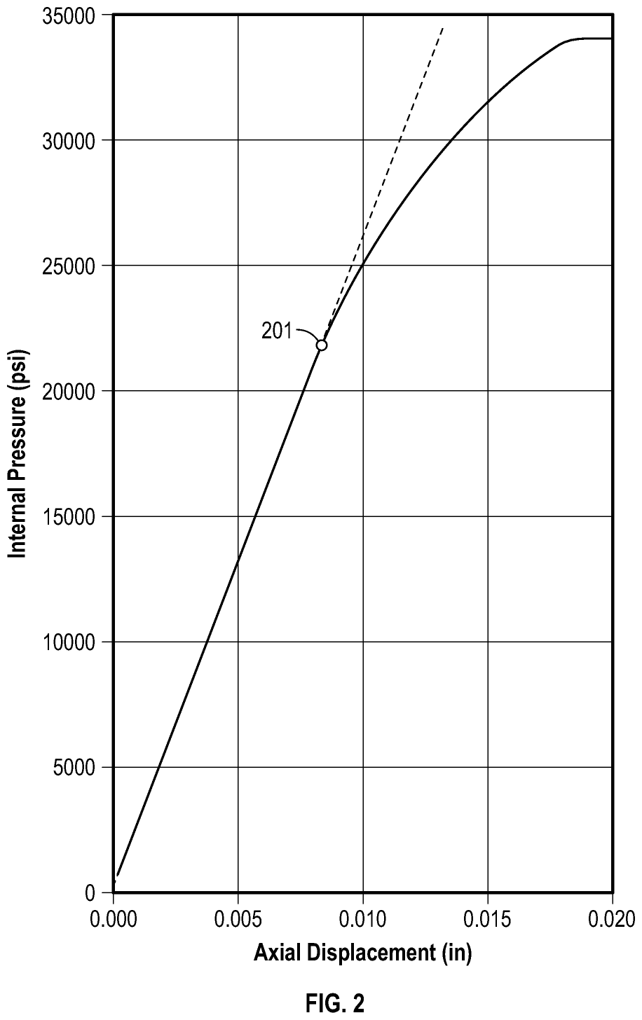

Beginning with excessive deformation, excessive deformation criterion is considered a service criterion, which limits potential risks leading to unsatisfactory performance of a component according to standards discussed in ASME VIII. Excessive deformation may be determined by finding an OGPD of a component, where excessive deformation is determined to occur. For example, FIG. 2 shows the load vs. displacement graph for a region of a threaded connection using ACME threads loaded by an internal pressure. The graph shows a linear relationship between load and displacement until an internal pressure of approximately 21,000 psi (i.e., 145 megapascals), and the relationship becomes non-linear after the internal pressure increases beyond 21,000 psi. The OGPD is determined to occur at the point 201, and after determining the OGPD (i.e., the point 201), a safety factor chosen for a selected operation may be applied to the OGPD to determine the working capability of the component. For example, if a safety factor is chosen to be 1.5 for an operation, the working capability of the component with the ACME threads may be computed by dividing the OGPD by the safety factor, which in this case is 21,000/1.5=14,000 psi (i.e., 97 megapascals). A safety factor may be selected based on, for example, the operation in which the component is to be used, the function of the component, and/or the location of the component, and may range, for example, from 1 to 3, or greater than 3. For example, a design engineer may select a relatively higher safety factor for components that are more critical for operation safety.

A design engineer may decide what constitutes excessive deformation and what is acceptable deformation of the component, interface, or assembly being analyzed. For example, some parts of a component or assembly may exhibit significant deformation and still be fit for purpose, whereas at the other extreme, some parts of a component or assembly may not have any or very little deformation in order to function. In the case of ACME threads, excessive deformation may be approximated to occur when one or more threads yields across its entire root. The working capability of the threads may be determined by applying a safety factor to the load at which OGPD occurs.

The capability of load bearing interfaces of a component (e.g., threads in a screw connection) may be defined by the excessive deformation criterion, because when non-linear stresses are incrementally applied (such as applied during simulation of the component), the load bearing interfaces exhibit incremental steps of gross plastic deformation before eventually reaching a global plastic collapse load. Due to the non-linearity of stepped gross plastic deformation, a judgement call may be used to identify the last valid load step that the component withstands before reaching the global plastic collapse load. This makes the analysis on fatigue life of a component relatively difficult, compared to cases with pressure vessel models (or other relatively perfect material testing samples) where transition from elasticity-dominated deformation to plasticity-dominated deformation occurs before reaching a clearly defined global plastic collapse load. In cases with continuously applied loads such as in a pressure vessel, the analysis for determining where transition from elasticity-dominated deformation to plasticity-dominated deformation occurs may be relatively simple, where the lastly converged load step that is valid may be determined without a judgement call.

The plastic collapse load is based on Load and Resistance Factor Design (LRFD) in which a model is incrementally loaded until the plastic collapse load is reached. The working capability based on LRFD may be determined by dividing the plastic collapse load by a selected load factor. For example, if the model is incrementally loaded to reach a plastic collapse load of 34,500 psi (i.e., 238 megapascals), and a load factor for an operating condition is approximated to be 1.11, the working capability of the component may be calculated as 34,500 psi/1.11=31,000 psi (i.e., 214 megapascals).

Embodiments of the present disclosure include methods for fatigue screening (i.e., fatigue assessment) of components used in oil and gas applications. Fatigue failures in oil and gas applications generally occur when the components are subject to alternating stresses below the static yield strength, over time. Cracks may initiate and then propagate in the weakest regions of the component (e.g., in the region with the highest strain), which leads to fatigue failures. Methods may include assessing the extent and flux of plastic deformation of a component as a result of cyclic internal pressure loading. If the extent and flux of plastic deformation is negligible then the component passes a test for fatigue screening. If the extent and flux of plastic deformation is significant then the component fails a test for fatigue screening.

In contrast to conventional SN methods for predicting material failure, fatigue screening methods disclosed herein may predict the fatigue life based on simulation results of an Onset of Gross Plastic Deformation (OGPD) of a plurality of regions of a component. In a threaded connection, for example, excessive deformation may be determined to occur at an OGPD, and the degree of plastic deformation may be approximated by analyzing certain parameters of the simulation results. For example, the degree of plastic deformation may be approximated from changes in gradient of a load vs. displacement graph generated from the simulation results. Small regions of inconsequential yielding typically do not affect the gradient of a load vs. displacement graph. However, as the load increases and the regions of yielding expand, the gradient of the graph may change. This change in gradient may be an indication of the OGPD.

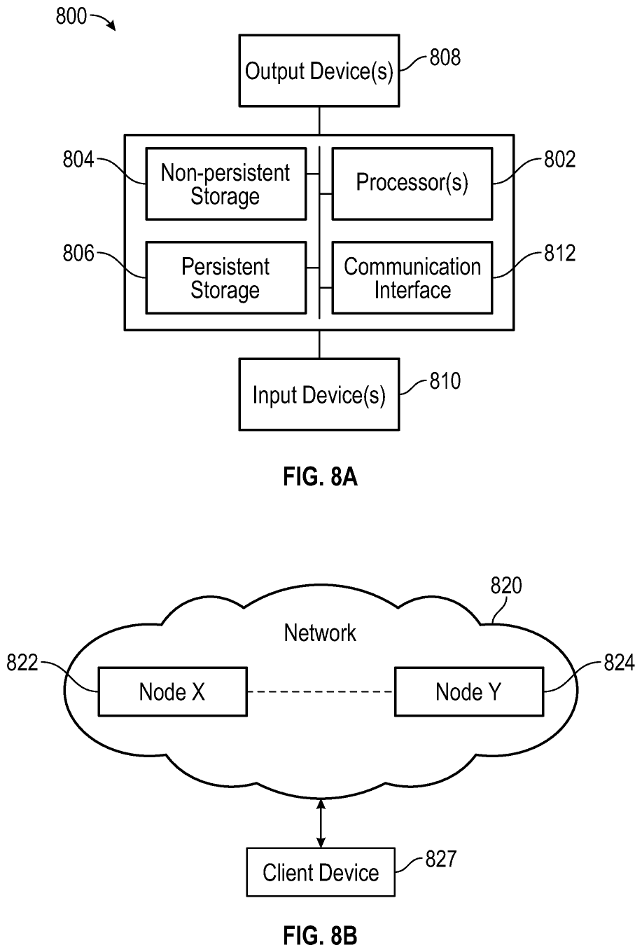

A method in accordance with one or more embodiments may include generating a 3D model of a component and its interfacing components using a computer aided design software in a computer-readable medium of a computer system. Such computer aided design software may provide an input file that comprises data for conditions of simulations of the model, as discussed in more detail below. The computer aided design software may be finite volume, finite difference, or FEA software such as ANSYS, ABAQUS, SolidWorks, and COMSOL, among others. For the computer system, any combination of mobile, desktop, server, router, switch, embedded device, or other types of hardware may be used. For example, as shown in FIG. 8A, the computing system 800 may include one or more computer processors 802, non-persistent storage 804 (e.g., volatile memory, such as random access memory (RAM), cache memory), persistent storage 806 (e.g., a hard disk, an optical drive such as a compact disk (CD) drive or digital versatile disk (DVD) drive, a flash memory, etc.), a communication interface 812 (e.g., Bluetooth interface, infrared interface, network interface, optical interface, etc.), and numerous other elements and functionalities.

The computer processor(s) 802 may be an integrated circuit for processing instructions. For example, the computer processor(s) may be one or more cores or micro-cores of a processor. The computing system 800 may also include one or more input devices 810, such as a touchscreen, keyboard, mouse, microphone, touchpad, electronic pen, or any other type of input device.

The communication interface 812 may include an integrated circuit for connecting the computing system 800 to a network (not shown) (e.g., a local area network (LAN), a wide area network (WAN) such as the Internet, mobile network, or any other type of network) and/or to another device, such as another computing device.

Further, the computing system 800 may include one or more output devices 808, such as a screen (e.g., a liquid crystal display (LCD), a plasma display, touchscreen, cathode ray tube (CRT) monitor, projector, or other display device), a printer, external storage, or any other output device. One or more of the output devices may be the same or different from the input device(s). The input and output device(s) may be locally or remotely connected to the computer processor(s) 802, non-persistent storage 804, and persistent storage 806. Many different types of computing systems exist, and the aforementioned input and output device(s) may take other forms.

Software instructions in the form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the disclosure.

The computing system 800 in FIG. 8A may be connected to or be a part of a network. For example, as shown in FIG. 8B, the network 820 may include multiple nodes (e.g., node X 822, node Y 824). Each node may correspond to a computing system, such as the computing system shown in FIG. 8A, or a group of nodes combined may correspond to the computing system shown in FIG. 8A. By way of an example, embodiments of the disclosure may be implemented on a node of a distributed system that is connected to other nodes. By way of another example, embodiments of the disclosure may be implemented on a distributed computing system having multiple nodes, where each portion of the disclosure may be located on a different node within the distributed computing system. Further, one or more elements of the aforementioned computing system 800 may be located at a remote location and connected to the other elements over a network.

Although not shown in FIG. 8B, the node may correspond to a blade in a server chassis that is connected to other nodes via a backplane. By way of another example, the node may correspond to a computer processor or micro-core of a computer processor with shared memory and/or resources.

The nodes (e.g., node X 822, node Y 824) in the network 820 may be configured to provide services for a client device 827. For example, the nodes may be part of a cloud computing system. The nodes may include functionality to receive requests from the client device 827 and transmit responses to the client device 827.

The computing system or group of computing systems described in FIGS. 8A and 8B may include functionality to perform a variety of operations disclosed herein. For example, the computing system(s) may perform communication between processes on the same or different systems. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file.

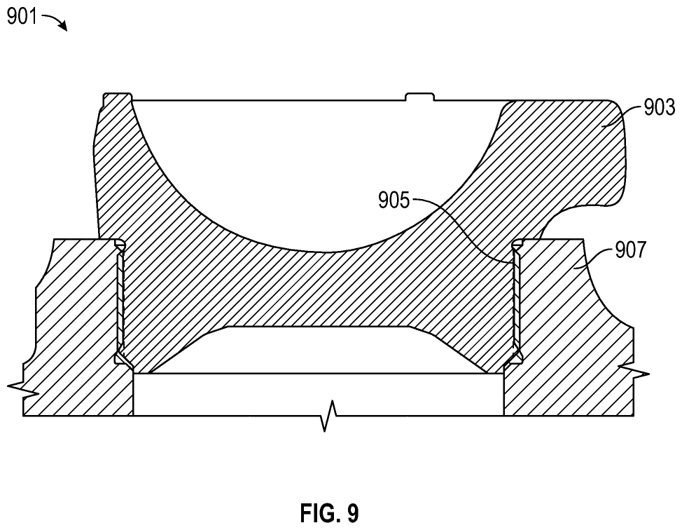

Such computing systems may be operated to perform simulations on components used in oil and gas applications. Examples of components may be pipes, valves, component connections and/or interfaces (e.g., threads), pumps, motors, manifolds, support structures, housings, pressure compensating devices, etc. As a non-limiting example, FIG. 9 shows a threaded cap connection 901 of a check valve in accordance with one or more embodiments that may include a cap 903 and threads 905 where the threads 905 may be chosen from a group of either one or combination of sharp, ACME, knuckle, square, and other conventionally known shapes of threads in order to avoid any slipping between the threads 905 and corresponding interfacing features 907. Threads 905 of such threaded cap connection 901 may be safeguarded against fatigue failures by preloading to such an extent that the cyclic stresses in the thread are sufficiently reduced.

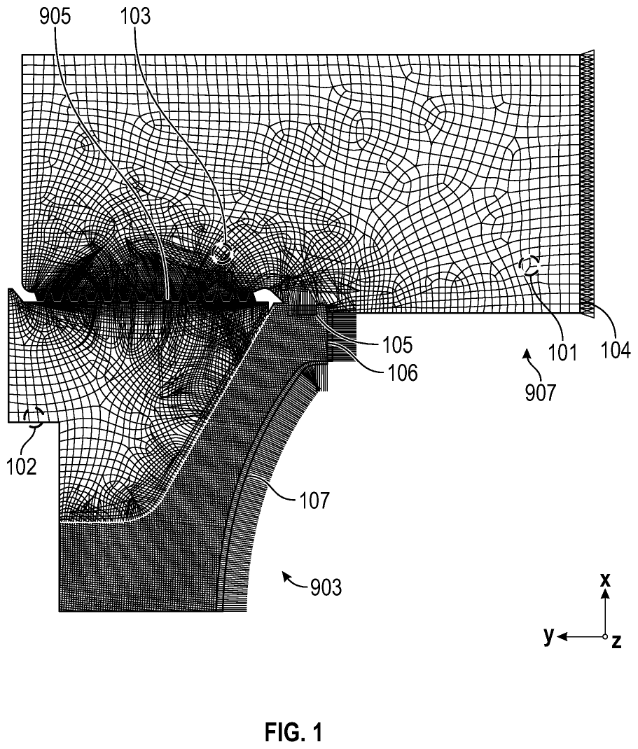

A 3D model of a component generated in accordance with one or more embodiments may include the component and its interfacing features written as an input file in a computer-readable medium of a computer system. The input file may include data describing the component and its interfacing features, such as size, shape, and material properties of the component, as well as the size/shape/design of the mesh used in the FEA to break up the component for analysis on a mesh element by element basis. For example, a 3D model of a component may include a mesh (e.g., polygonal grids) overlaid onto the model of the component to delineate a plurality of mesh elements of the component, where a simulation of the component may analyze each mesh element. The aggregate of each elemental analysis may provide an overall analysis of the performance of the component during the simulation. A mesh may define a plurality of differently sized and shaped mesh elements including multiple nodal points at the vertices of the mesh elements. For example, when a mesh defines a plurality of polygonal mesh elements having straight sides and/or curved sides and at least one vertex, each nodal point is located at the vertices of the polygon mesh elements formed during meshing of the model. Types of grid structures and the mesh elements may be chosen upon a designer's selection. For example, FIG. 1 shows a cross-sectional view of a region of the component in FIG. 9. The body 903 and its corresponding interfacing component 907 are generated in a computer-coded 3D model where the model has a mesh overlay delineating the modeled component regions into a plurality of differently sized and shaped mesh elements 101 including a number of nodal points. The mesh may be sized differently based on the amount of stress an area of a component is expected to experience during simulation.

For example, FIG. 1 shows the mesh overlay defined differently on a plurality of regions. Specifically, relatively large mesh elements 102 are used in areas that are expected to experience minimal stress. In areas where large stress is expected to occur, such as areas of the threads 905 and the interfacing component 907, relatively smaller mesh elements 103 (i.e. finer mesh) are used in order to efficiently use the computer resources and obtain accurate results of the simulations. 3D models in accordance with other embodiments may also include other regions that are deemed critical. In some embodiments, a plurality of regions generated in a 3D model of a check valve may include at least both of the longitudinal ends and the middle regions of the body and the threads of the threaded connection. Once the model is created as an input file in the computer-readable medium, a computer aided design software, such as ANSYS, may be used to perform simulations on the model to determine an OGPD in the plurality of regions. It is possible to analyze the input file of the model using a design software that is different from the one used to write the input file.

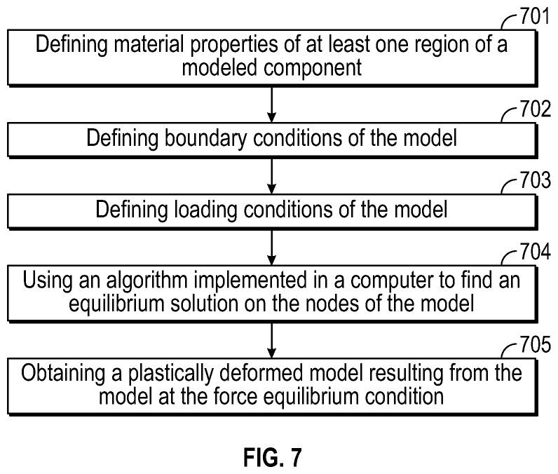

FIG. 7 shows a flow chart of a method for performing simulations on a component under a high cycling internal pressure loading in accordance with one or more embodiments. The flow chart shows a plurality of steps taken in order to determine an OGPD in a plurality of regions of a component (e.g., a threaded connection region). Beginning with step 701, material properties, such as density, Young's Modulus, ultimate strength, yield point, strength, hardness, corrosion resistance, ductility, elasticity/stiffness, fracture toughness, plasticity, impact resistance, and other parameters are defined for at least one region of a modeled component. Material properties inputted into the FEA software may include any parameter that may be used to describe a characteristic of the material forming the component being simulated. In some embodiments, material properties may be defined and inputted into the FEA software for a body, interfacing features, and threads of the modeled component. In some embodiments, material properties may be defined and inputted into the FEA software for the entire modeled component. In some embodiments, material properties may be defined and inputted for at least one region of a modeled component and at least one interfacing region of an interfacing component.

At steps 702 and 703, boundary conditions and loading conditions of the model are defined, respectively. If the simulations are performed on a particular region of a component, boundary nodal points may be defined around the particular region (e.g., boundaries of the simulated region of the component), as shown in points 104 in FIG. 1.

Boundary conditions defined and inputted into the FEA software may include, for example, the location of the boundary of the region(s) of the modeled component to be simulated (e.g., the location of the boundary nodal points and/or location of the mesh elements around the boundary of the portion of the modeled component) and characteristics of elements interfacing the boundary (e.g., type and material of interfacing components around the boundary and/or the environment interfacing around the boundary such as seawater/air).

Loading conditions defined and inputted into the FEA software may include values and types of forces acting on the region(s) of the modeled component being simulated, such as pressure, temperature, and loading from adjacent regions and/or components, as well as characteristics of the material reaction to forces acting on the region(s) of the modeled component being simulated, such as deformation patterns (e.g., slip directions for dislocation).

For example, referring to FIG. 1, points 105, 106, 107 correspond to loads that are defined for a loading condition of the component subjected to a working condition being simulated. Stationary nodal points that do not move during simulations are fixed when the boundary conditions are defined. Initial conditions, such as initial working loads, and deformation patterns of the model may be defined when the loading conditions are defined at step 703. All of the aforementioned conditions for the simulation may be saved as data in input files.

An equilibrium solution on the nodal points of a model may be obtained at step 704 using an algorithm implemented on the computing system. The equilibrium solution may include a force equilibrium obtained on the nodal points after the boundary conditions and the loading conditions are applied. At step 705, the force equilibrium condition applied to the simulated model results into a plastically deformed model of the component.

As the working conditions of the component is being simulated, including simulated loading conditions and simulated component behavior from the equilibrium solution, displacement (e.g., dislocation, movement or other change in size/shape) of the simulated region(s) of the component may be recorded as a function of the amount of load applied during the simulation. For example, relative displacement of a particular nodal point of a meshed element in the simulation results may be analyzed with respect to an initial position of the nodal point, providing data for the displacement and the load applied to the nodal point. From the recorded load and displacement data, a load vs. displacement graph may be generated. The generated load vs. displacement graph may be used to determine the OGPD of the simulated region(s) of the component, as described herein.

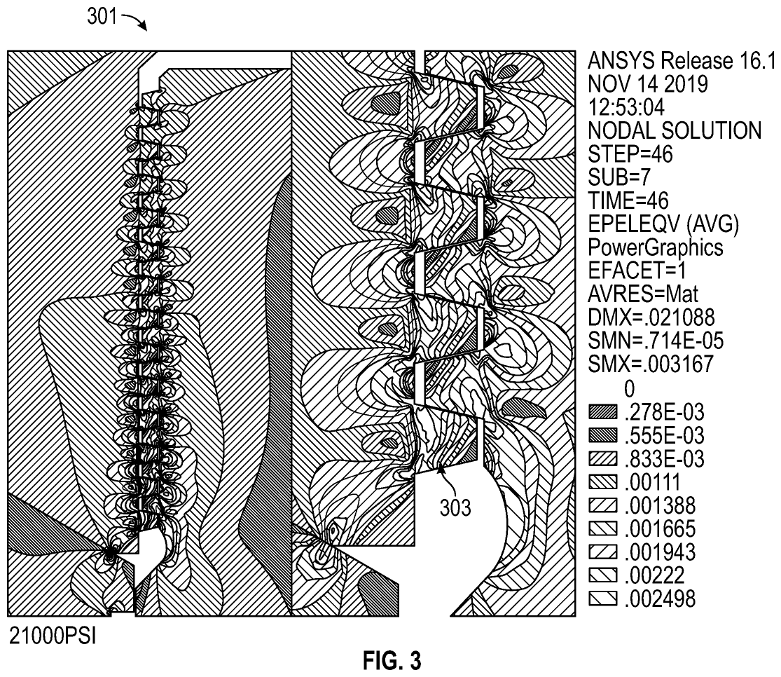

If a load vs. displacement graph does not have a clear transition from linear to nonlinear, or if more resolution is required in identifying the OGPD, Elastic Strain plots may be used to help identify the OGPD. FIG. 3 shows an Elastic Strain Plot of a threaded connection between two components in accordance with one or more embodiments, when an internal pressure on the threaded connection is 21,000 psi (i.e., 145 megapascals). The model on the left shows a cross sectional view of a threaded connection 301, and the model on the right shows a magnified view of the strains on the teeth 303 of the threaded connection 301. A maximum contour value is calculated as the yield strength divided by Young's Modulus multiplied by a model parameter (e.g., 0.999×yield strength/Young's Modulus). The maximum contour value may be used to approximate the extent of plastic deformation when using an elastic-perfectly plastic material model. For example, FIG. 3 shows a significant extent of plastic deformation as regions around the teeth 303 indicate high concentration of stress.

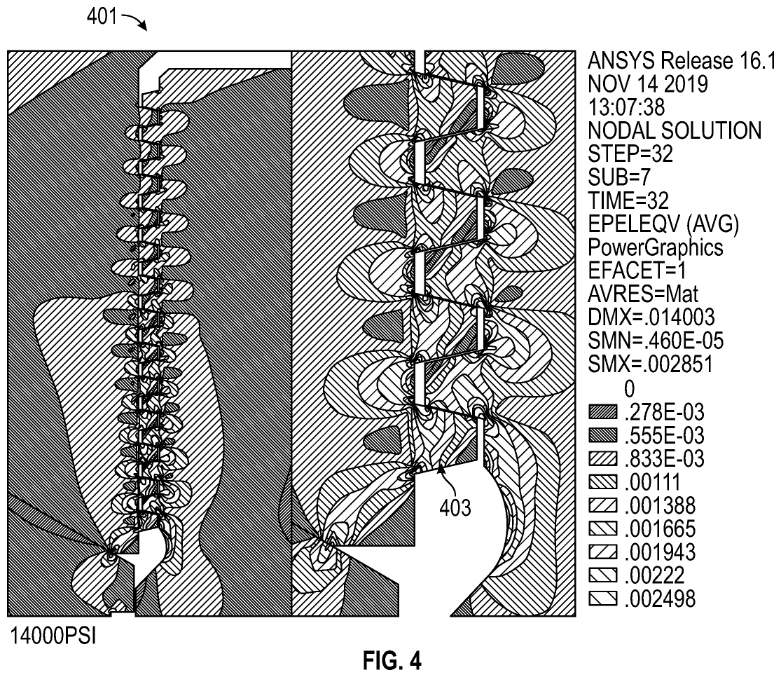

FIG. 4 shows a simulation result when an internal pressure is less than 21,000 psi (i.e., 145 megapascals) where the model on the left shows a cross-sectional view of a modeled threaded connection 401 between two interfacing components, and the model on the right shows a magnified view of the strains on the teeth 403 of the threaded connection 401. The OGPD is deemed to occur at an approximate internal pressure of 21,000 psi and that the working capability of the ACME threads is computed as 21,000/1.5=14,000 psi (i.e., 97 megapascals).

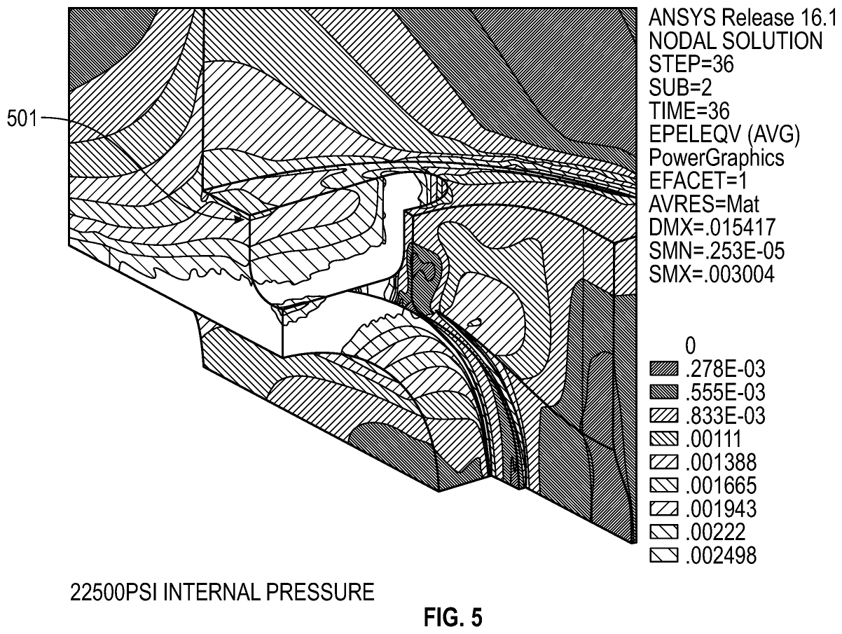

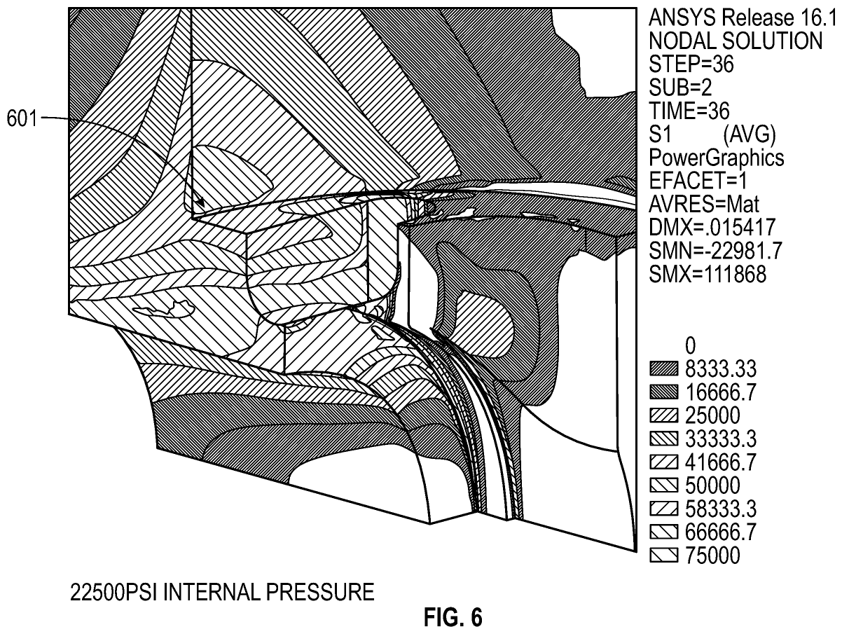

Performing simulations on a model of an assembly or component, for example, a check valve, in accordance with one or more embodiments may include performing simulations on a plurality of regions of the model that includes interfacing feature 501, 601, as shown in FIGS. 5 and 6, respectively. This interfacing feature 501, 601 has gross plastic deformation beginning at around 15,000 psi (i.e., 103 megapascals). However, FIG. 6 shows that at 22,500 psi (i.e., 155 megapascals), the first principal stress does not exceed the yield of the material, suggesting that much of the loads in this region put the material into compression. For this reason, these interfacing features are considered to be resistant against fatigues due to pressure cycles up to, and possibly beyond, 15,000 psi. One skilled in the art would appreciate how the method disclosed herein may determine if a region of a model of an assembly or component is fatigue resistant based on the OGPD, not based on any conventionally known methods, such as stress-life (SN) approach.

Modeled components may include one or more bodies having a load bearing interface surface that may be subjected to a pressure or load during operation of the component. For example, component models may include multiple bodies having at least one load bearing interface there between. Simulations on modeled components may include simulating different loads on at least the regions including and surrounding the load bearing interface surfaces to determine how the selected areas react to the applied loads. Modeled components may include, but are not limited to, valves and valve part connections, connected-together components (e.g., connected tubulars), fasteners, and other components that may be operational under cyclical or consistent loads (e.g., operated under an elevated pressure), for example.

Fatigue analysis methods according to embodiments of the present disclosure may be particularly useful in oil and gas applications, where equipment may be subjected to large amounts of cyclical pressure in unique environments (e.g., subsea), and where equipment reliability may be needed for safety reasons (e.g., pressure relief elements and sealing elements).

Methods disclosed herein for determining an OGPD may be used on different components, which may be used for predicting the operational life of each component. Further, by using methods disclosed herein, the operational life of a component may be predicted based on one or more simulations of the component, which may be completed, for example, in less than a day. In contrast, conventional methods of predicting an operational life of a component, which may have included using material stress-strain testing and/or analysis of failed components from the field, for example, may take many days or years in the case of failed field component analysis.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed:

1. A method comprising:
generating a 3D computer-coded model of a component; and
performing simulations on the model to determine an onset of gross plastic deformation in a plurality of regions of the component;
wherein the model is stored in a computer-readable medium.

2. A method of claim 1, wherein the model of the component comprises:
a body; and
a load bearing interface, wherein the load bearing interface is designed to contain pressure or support a load;
wherein the simulations are performed at different pressures or loads on the load bearing interface.

3. The method of claim 1, wherein the component is a threaded connection with threads chosen from a group of either one or combination of sharp, ACME, knuckle, square, and other conventionally known shapes of threads.

4. A method of claim 3, wherein the plurality of regions comprises at least both of longitudinal ends and middle regions of the threads of the component.

5. The method of claim 1, wherein generating the model comprises delineating a mesh overlaid onto the model, wherein the mesh defines a plurality of mesh elements and nodal points at vertices of the mesh elements.

6. The method of claim 5, wherein the performing simulations comprises:
defining material properties of the component;
defining boundary conditions of the model;
defining loading conditions on the model; and
using an algorithm implemented in a computer to find an equilibrium solution on the nodal points of the model;
wherein the equilibrium solution comprises a force equilibrium of the nodal points of the model in the boundary conditions and the loading conditions; and
wherein the model at the force equilibrium condition results into a plastically deformed model.

7. The method in claim 6, wherein defining boundary conditions further defines stationary nodal points that are fixed during the simulations.

8. The method in claim 6, wherein defining loading conditions further defines initial conditions, working loads, and deformation patterns of the simulations of the model.

9. The method of claim 1, wherein the onset of gross plastic deformation is defined when a parameter across a region exceeds a maximum contour value obtained by an equation, wherein the region comprises the highest stress concentration.

10. The method of claim 9, wherein the parameter is a change in slope of a stress-strain curve during simulations.

11. A method comprising:
determining an onset of gross plastic deformation of a component by performing simulations on a 3D computer-coded model of a plurality of regions of the component; and
dividing the onset of gross plastic deformation by a safety factor to calculate a working capability load of the model.

12. The method of claim 11, wherein the model of the component comprises:
multiple bodies; and
at least one load bearing interface between the multiple bodies.

13. The method of claim 12, wherein the plurality of regions comprises at least surfaces of the multiple bodies forming the at least one load bearing interface.

14. The method in claim 12, wherein the at least one load bearing interface comprises a threaded connection with threads chosen from a group of either one or combination of sharp, ACME, knuckle, square, and other conventionally known shapes of threads.

15. The method in claim 12, wherein the performing simulations comprises:
defining material properties of the multiple bodies;
defining boundary conditions of the model;
defining loading conditions of the model;
using an algorithm implemented in a computer to find an equilibrium solution on nodal points defined on the model;
wherein the equilibrium solution comprises a force equilibrium of the nodal points of the model in the boundary conditions and the loading conditions; and
wherein the model at the force equilibrium condition results into a plastically deformed model.

16. The method in claim 15, wherein defining boundary conditions further defines stationary nodal points that are fixed during the simulations.

17. The method in claim 15, wherein defining loading conditions further defines initial conditions, working loads, and deformation patterns of the simulations of the model.

18. The method of claim 11, wherein the onset of gross plastic deformation is defined when a parameter across a region exceeds a maximum contour value obtained by an equation, wherein the region comprises the highest stress concentration.

19. The method of claim 18, wherein the parameter is a change in slope of a stress-strain curve during simulations.

20. The method of claim 11 further comprises determining if the model is safe under a load by comparing all of onset of gross plastic deformation of the plurality regions of the component.
